(19) United States
(12) Patent Application Publication
(10) Pub. No.: US 2025/0282867 A1
(43) Pub. Date: Sep. 11, 2025

(54) Pharmaceutical Composition for Preventing or Treating Sjogren's Syndrome Comprising Anti-SEMA4D Blocking Antibody as Active Ingredient

(71) Applicant: SIALBIO CO., LTD., Seoul (KR)

(72) Inventors: Sangwoo Lee, Seoul (KR); Kyung Pyo Park, Seoul (KR)

(21) Appl. No.: 19/213,610

(22) Filed: May 20, 2025

Related U.S. Application Data
(63) Continuation-in-part of application No. PCT/KR2024/004245, filed on Apr. 2, 2024.

(30) Foreign Application Priority Data
Apr. 3, 2023 (KR) 10-2023-0043767
Jan. 12, 2024 (KR) 10-2024-0005193

(51) Int. Cl. C07K 16/28; A61K 9/00; A61K 39/00; A61P 37/06; C07K 16/24

(52) U.S. Cl. CPC C07K 16/2803; A61K 9/0019; A61P 37/06; C07K 16/24; A61K 2039/505; A61K 2039/54; C07K 2317/24; C07K 2317/76

(57) ABSTRACT

Provided is a pharmaceutical composition for preventing or treating Sjogren's syndrome including an anti-SEMA4D blocking antibody as an active ingredient.

### CROSS-REFERENCE TO RELATED APPLICATION

This application is a continuation-in-part application of PCT/KR2024/004245 filed Apr. 2, 2024, which claims priority to Korean Patent Application Nos. 10-2023-0043767 filed Apr. 3, 2023 and 10-2024-0005193 filed Jan. 12, 2024, of which the contents are incorporated in their entireties by reference.

### FIELD

The present disclosure relates to a pharmaceutical composition for preventing or treating Sjogren's syndrome including an anti-Semaphorin 4D (SEMA4D) blocking antibody as an active ingredient.

### BACKGROUND

Sjogren's syndrome is a rare autoimmune disease of unknown etiology, characterized by decreased secretion of lachrymal glands and salivary glands, resulting in keratoconjunctivitis and xerostomia. For reasons that are not yet elucidated, autoimmune antibodies are produced and bind to exocrine tissues, such as salivary glands and lachrymal glands, thereby inducing infiltration of immune cells, including T cells and B cells, into the exocrine organs, causing a severe inflammatory response and ultimately leading to destruction of the tissues. Salivary dysfunction in Sjogren's syndrome progressively worsens, and salivary gland function is rarely restored, such that patients suffer from persistent xerostomia throughout life.

Meanwhile, there is yet disclosed no fundamental treatment for Sjogren's syndrome, and symptomatic treatment includes taking immunosuppressants such as prednisolone or prescribing pilocarpine, which artificially increases the amount of saliva by stimulating the parasympathetic nerves. However, when pilocarpine is applied, pilocarpine activates muscarinic receptors throughout the body to cause various side effects such as heart palpitations, dizziness, and hyperhidrosis, and is just a temporary expedient that acinar cells that produce saliva are continuously destroyed, the destruction of the salivary glands is not prevented, and the remaining salivary gland acinar cells are stimulated to produce saliva as Sjogren's syndrome progresses. To this end, there are patients who do not experience a significant increase in a saliva amount even when taking pilocarpine, and in these cases, there are applied methods of replacing the function of saliva by using artificial saliva.

Development of salivary gland regenerative therapy for Sjogren's syndrome is challenging, as even if functional regeneration is achieved, a vicious cycle may occur in which regenerated salivary gland cells are again targeted by circulating autoimmune antibodies, leading to excessive infiltration of T cells and B cells and subsequent destruction of the regenerated tissues. Ultimately, the salivary dysfunction observed in Sjogren's syndrome is caused by various cytokines secreted by excessively activated immune cells infiltrating into the salivary gland tissues, and no therapeutic modality capable of inhibiting this pathological process has been established. Therefore, there is an urgent need to develop a therapeutic strategy capable of suppressing immune cell infiltration and migration into the tissues and regulating immune cell activation.

### PRIOR ART DOCUMENT

KR 10-2312956 (Oct. 7, 2021)
KR 10-2022-0011652 (Jan. 28, 2022).

### Technical Problem

An object of the present disclosure is to provide a method for preventing or treating Sjogren's syndrome, the method comprising locally injecting an anti-Semaphorin 4D (SEMA4D) blocking antibody into a salivary gland of a patient in order to inhibit immune cell infiltration into the salivary gland induced by Sjogren's syndrome, thereby restoring salivary gland function.

Another object of the present disclosure is to provide a pharmaceutical composition for preventing or treating Sjogren's syndrome including an anti-SEMA4D blocking antibody as an active ingredient.

Still another object of the present disclosure is to provide a diagnostic kit for Sjogren's syndrome, the kit comprising a SEMA4D antibody and a CXCL12 antibody.

### Technical Solution

In order to achieve the objects, an aspect of the present disclosure provides a pharmaceutical composition for preventing or treating Sjogren's syndrome comprising an anti-SEMA4D blocking antibody as an active ingredient.

The anti-SEMA4D blocking antibody may inhibit the expression level of at least one gene selected from the group consisting of CXCL12, CXCR4, and a combination thereof.

In addition, the anti-SEMA4D blocking antibody may block at least one protein selected from the group consisting of CD72, CD100, Plexin B2, and a combination thereof.

In addition, the pharmaceutical composition may be formulated for administration by injection.

In addition, the pharmaceutical composition may be administered to a subject via local administration into a salivary gland.

Yet another aspect of the present disclosure provides a diagnostic kit for Sjogren's syndrome, the kit comprising at least one antibody selected from the group consisting of a SEMA4D antibody, a CXCL12 antibody, and a combination thereof, as an active ingredient.

Still another aspect of the present disclosure provides a diagnostic kit for Sjogren's syndrome, the kit comprising a SEMA4D antibody and a CXCL12 antibody.

### Advantageous Effects

According to embodiments of the present disclosure, the pharmaceutical composition for preventing or treating Sjogren's syndrome and the use thereof can alleviate hyposalivation by administering an anti-SEMA4D blocking antibody, thereby effectively inhibiting the migration of immune cells responsible for the destruction of salivary glands.

In addition, the pharmaceutical composition for preventing or treating Sjogren's syndrome of the present disclosure may be formulated for administration by injection, thereby minimizing systemic side effects such as heart palpitations, dizziness, and hyperhidrosis.

In addition, the pharmaceutical composition of the present disclosure may be administered via local injection into a salivary gland, thereby achieving a significant therapeutic effect with a smaller dosage compared to systemic administration.

In addition, the diagnostic kit for detecting SEMA4D and CXCL12 according to the present disclosure enables the diagnosis of Sjogren's syndrome with high accuracy and sensitivity.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1A is a schematic diagram illustrating a pathological phenomenon in Sjogren's syndrome; and FIGS. 1B to 1F show results of analyzing the expression levels of SEMA4D in a normal group and a patient group with Sjogren's syndrome;

FIGS. 2A to 2G are graphs showing analyzing a correlation between soluble SEMA4D (sSEMA4D) and CXCL12 concentrations in the saliva and serum of patients with Sjogren's syndrome;

FIG. 3A is a schematic diagram illustrating a co-culture system of salivary gland epithelial cell-derived cell lines or organoids with Jurkat cells, a human CD4⁺ T cell line;

FIGS. 3B and 3C show results of analyzing effects of recombinant SEMA4D (rS4D) and an anti-SEMA4D blocking antibody (aS4D) in co-culturing system;

FIGS. 4A to 4C show results of analyzing effects of recombinant SEMA4D (rS4D) on salivary gland-derived epithelial cells;

FIGS. 5A and 5B show results of analyzing receptors bound by SEMA4D under the influence of CXCL12 in salivary gland-derived organoids;

FIGS. 6A to 6E show results of analyzing the effects of SEMA4D on T cells;

FIGS. 7A to 7E show results of analyzing the symptoms of Sjogren's syndrome in NOD mice, a mouse model of Sjogren's mouse;

FIGS. 8A to 8C show results of analyzing an administration route for treating Sjogren's syndrome;

FIGS. 9A to 9G show results of treating Sjogren's syndrome by an anti-SEMA4D blocking antibody; and

FIGS. 10A and 10B show results of treating Sjogren's syndrome by various types of anti-SEMA4D blocking antibodies.

### DETAILED DESCRIPTION

Hereinafter, embodiments of the present disclosure will be described in detail. However, the present disclosure is not limited to the embodiments set forth herein, and may be embodied in various modifications and alternative forms. The embodiments are provided merely to fully convey the scope of the present disclosure to those skilled in the art.

As used herein, the term “treatment” refers to any therapeutic intervention that cures or alleviates symptoms of a diagnosed pathological condition or disorder such as Sjogren's syndrome, or delays or halts the progression thereof. The term also encompasses prophylactic or preventive measures that prevent or delay the onset of such a condition or disorder.

As used herein, the term “prevention” refers to all actions that inhibit or delay the occurrence, spread, or recurrence of Sjogren's syndrome through the administration of the pharmaceutical composition of the present disclosure.

As used herein, the symptoms of treatment, alleviation, or improvement of Sjogren's syndrome include, but are not limited to: inhibition of immune cell infiltration into salivary gland tissue; restoration of saliva secretion to normal levels; reduction in the number of inflammatory foci within the salivary gland tissue or a reduction in the number of T cells infiltrating such foci; regeneration of destroyed salivary gland tissue; and reduction in the level of CXCL12.

As used herein, the terms “subject,” “individual,” or “patient” refer to a biological entity to which the pharmaceutical composition of the present disclosure is administered, and more particularly, a human subject.

As used herein, the term “diagnosis” includes determining whether an individual is susceptible to a disease or disorder, determining the presence of the disease or disorder, assessing the prognosis of an individual diagnosed with the disease, or monitoring the disease state or response to treatment.

As used herein, the terms “anti-SEMA4D antibody” and “SEMA4D antibody” are used interchangeably. The SEMA4D antibody (anti-SEMA4D antibody) may be a polyclonal antibody, a monoclonal antibody, or functional fragments thereof. Among them, the monoclonal antibody is preferred due to high antigen specificity. However, when the antibody is applied to a diagnostic kit, the SEMA4D antibody may be selected from a polyclonal antibody, a monoclonal antibody, or a functional fragment thereof without limitation.

The antibody that binds to SEMA4D may be an antigen-binding fragment, a variant, or a derivative thereof.

The SEMA4D also referred to as CD100, is a transmembrane protein, expressed in immune cells such as T cells and B cells as well as certain tumor cells. The protein is cleaved by proteases such as matrix metalloproteinases (MMP) and a disintegrin and metalloprotease (ADAM) to form a soluble form (sSEMA4D or sS4D), which plays an important role in the immune system, nervous system, and tumor microenvironment.

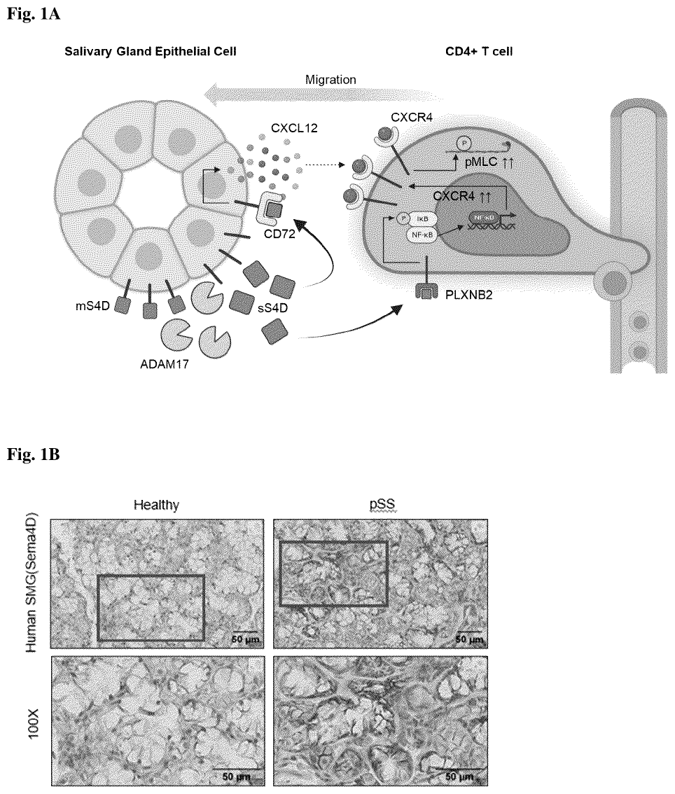

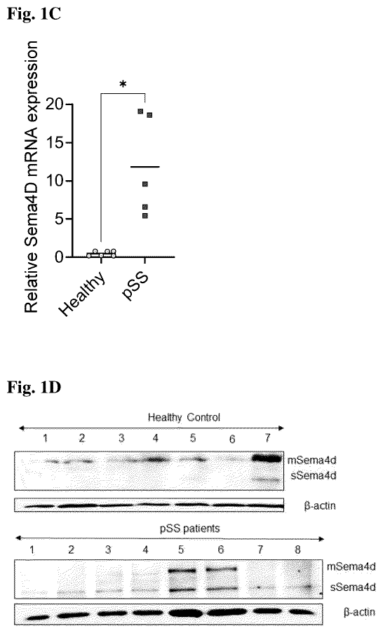

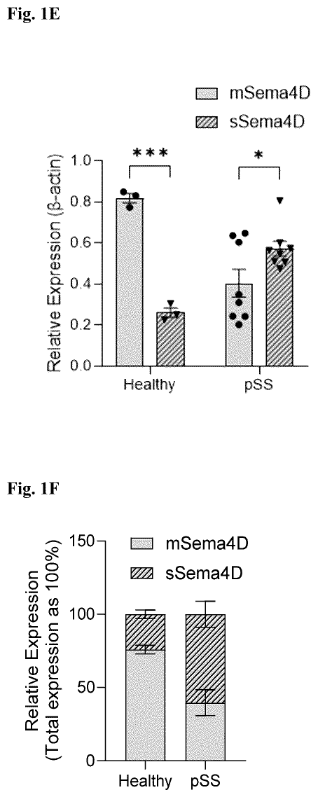

In the embodiment, it was confirmed that the expression level of SEMA4D is elevated in salivary gland epithelial tissues of patients with Sjogren's syndrome, and a large amount of soluble sSEMA4D is present therein. As illustrated in FIG. 1A, excessive sSEMA4D binds to Plexin B2 in salivary gland epithelial cells to induce overexpression of CXCL12, and also binds to Plexin B2 and CD72 in CD4⁺ T cells to activate NF-κB signaling, thereby inducing the overexpression of CXCR4, a receptor of CXCL12. That is, a synergistic effect between the overproduced CXCL12 in epithelial cells and the overexpressed CXCR4 in CD4⁺ T cells enhances immune cell infiltration into epithelial cells (see FIG. 1A).

In the embodiment, it was confirmed that administration of the anti-SEMA4D blocking antibody effectively inhibited the infiltration of immune cells into salivary gland epithelial tissues, thereby alleviating hyposalivation. Specifically, the anti-SEMA4D blocking antibody may inhibit the expression of one or more genes selected from the group consisting of CXCL12, CXCR4, or a combination thereof by blocking one or more proteins selected from the group consisting of CD72, CD100, Plexin B2, or a combination thereof.

Furthermore, in another embodiment, it was confirmed that direct administration of recombinant soluble SEMA4D (rSEMA4D) into the salivary gland of a normal mouse resulted in Sjogren's syndrome-like symptoms, including reduced saliva secretion, increased CXCL12 expression, and enhanced infiltration of immune cells into the salivary gland tissue. These results suggest that elevated SEMA4D expression in salivary gland tissues constitutes a pathogenic factor in Sjogren's syndrome, and that early therapeutic intervention with an antagonist, such as an anti-SEMA4D blocking antibody, may prevent the onset or progression of the disease.

The SEMA4D antibody comprised in the pharmaceutical composition of the present disclosure may be a blocking antibody that inhibits downstream signaling by binding to soluble SEMA4D (sSEMA4D), thereby preventing the interaction between sSEMA4D and Plexin B1. The anti-SEMA4D blocking antibody included in the pharmaceutical composition for the prevention or treatment of Sjogren's syndrome may specifically bind to sSEMA4D of human origin, rodent origin, or both. The antibody may be selected from the group consisting of mouse antibodies, rat antibodies, human antibodies, humanized antibodies, chimeric antibodies, or antigen-binding fragments thereof. As used herein, the terms “blocking antibody” and “antagonist antibody” are used interchangeably.

Anti-SEMA4D blocking antibody is already known in the art. Non-limiting examples include those described in U.S. Pat. No. 8,496,938 B2; U.S. Patent Application Publication Nos. 2008/0219971 A1, 2010/0285036 A1, and 2006/0233793 A1; International Patent Publications WO 93/14125 A1, WO 2008/100995 A1, and WO 2010/129917 A2; and Herold et al., Int. Immunol. 7 (1): 1-8 (1995). Commercially available examples of anti-SEMA4D blocking antibodies include Pepinemab (VX15/2503, Vaccinex), BMA-12 (eBioscience), and C100 (C-3) (sc-390675, Santa Cruz Biotechnology).

The complementarity-determining regions (CDRs) 1 to 3 of the heavy chain variable region of Pepinemab (VX15/2503, Vaccinex) are GYSFSDYYMH (CDR1), QINPTTGGASYNQKFKG (CDR2), and YYYGRHFDV (CDR3), respectively. The CDRs 1 to 3 of the light chain variable region thereof are KASQSVDYDGDSYMN (CDR1), AASNLES (CDR2), and QQSNEDPYT (CDR3), respectively.

The pharmaceutical composition for preventing or treating Sjogren's syndrome according to the present disclosure may be administered in the form of an injection for local delivery to the salivary gland. In this case, the pharmaceutical composition may be formulated in unit dosage ampoules or multi-dose forms by combining a pharmaceutically acceptable diluent with one or more of a preservative, isotonic agent, analgesic, solubilizer, buffer, or stabilizer. Accordingly, systemic adverse effects such as palpitations, dizziness, and hyperhidrosis may be reduced.

When the pharmaceutical composition of the present disclosure is administered via local injection into the salivary gland for the treatment of Sjogren's syndrome, a superior therapeutic effect—such as restoration of salivary secretion and inhibition of inflammatory cell infiltration—may be achieved in comparison to systemic administration (e.g., intraperitoneal or intravenous injection). Local injection enables a higher local drug concentration to be maintained, thereby allowing effective treatment with a lower dosage.

Accordingly, the present disclosure provides a method for preventing or treating Sjogren's syndrome, comprising locally administering the pharmaceutical composition of the present disclosure into the salivary gland of a subject in need thereof.

The pharmaceutical composition of the present disclosure may further comprise one or more pharmaceutically acceptable additives, such as carriers, diluents, binders, disintegrants, lubricants, pH regulators, antioxidants, or solubilizers, within a range that does not interfere with the activity of the active ingredient. Examples of such pharmaceutically acceptable additives include, but are not limited to, microcrystalline cellulose, xylitol, erythritol, methylcellulose, polyvinylpyrrolidone, starch, acacia, alginate, gelatin, lactose, dextrose, sucrose, propylhydroxybenzoate, cellulose, water, methylhydroxybenzoate, magnesium stearate, talc, sorbitol, mannitol, maltitol, calcium phosphate, calcium silicate, and mineral oil.

The amount of the active ingredient contained in the pharmaceutical composition according to the present disclosure may be appropriately determined in consideration of factors such as the absorption rate, inactivation rate, and excretion rate of the active ingredient in vivo, as well as the age, sex, and health condition of the subject. The active ingredient may be administered at a concentration ranging from 0.0001 nM to 1000 mM, preferably from 0.001 nM to 100 mM, and may be administered once to three times per day or multiple times over a predetermined period.

The present disclosure further provides a diagnostic kit for diagnosing Sjogren's syndrome. The diagnostic kit may comprise, as an active component, a SEMA4D antibody, a CXCL12 antibody, or a combination thereof. The diagnosis of Sjogren's syndrome may be performed by quantifying the levels of SEMA4D and/or CXCL12 in a biological sample, such as saliva or blood, using an immunoassay such as ELISA. The SEMA4D antibody included in the diagnostic kit of the present disclosure is capable of binding to sSEMA4D, but unlike the anti-SEMA4D blocking antibody included in the pharmaceutical composition of the present disclosure, it is not required to inhibit downstream signaling of SEMA4D.

The diagnostic kit for Sjogren's syndrome according to the present disclosure enables the diagnosis of whether a subject is afflicted with Sjogren's syndrome by measuring and analyzing the concentration of sSEMA4D or both sSEMA4D and CXCL12 in the subject's saliva. Since the diagnostic method employs saliva rather than blood, it provides the advantage of being non-invasive while still achieving a high level of diagnostic accuracy.

In particular, when both sSEMA4D and CXCL12 markers are used in saliva, the area under the curve (AUC) reaches 0.967, whereas the AUC is 0.867 when using sSEMA4D alone and 0.861 when using CXCL12 alone. Accordingly, simultaneous analysis of both sSEMA4D and CXCL12 enables the diagnosis of Sjogren's syndrome with superior sensitivity and specificity.

Hereinafter, Examples will be provided to further illustrate the present disclosure in greater detail. However, these Examples are merely intended to exemplify the present disclosure and shall not be construed as limiting the scope thereof.

### EXAMPLES

### Example 1: Analysis of Salivary Gland Tissue from Patients with Sjogren's Syndrome

To identify the characteristics of patients with Sjogren's syndrome, the distribution of SEMA4D in the salivary glands of a control group and patients with Sjogren's syndrome was analyzed by immunohistochemistry using anti-SEMA4D antibodies on biopsy tissues from the salivary glands of healthy individuals (control group) and patients with Sjogren's syndrome (pSS). Additionally, the distribution of full-length SEMA4D (mSEMA4D) and soluble SEMA4D (sSEMA4D) was analyzed by Western blotting. The antibody used for this analysis was sc-390675 from Santa Cruz. Furthermore, the expression level of SEMA4D mRNA was analyzed using RT-PCR.

Referring to FIGS. 1B to 1G, SEMA4D was expressed at high levels in both protein and mRNA levels in salivary gland epithelial cells of the patients with Sjogren's syndrome (FIGS. 1B and 1C). Notably, sSEMA4D could be distinguished in the results, and its presence in the salivary gland tissues of the control group and patients with Sjogren's syndrome was analyzed by Western blotting. As a result, sSEMA4D was barely detectable in the salivary glands of the control group, whereas in the salivary glands of patients with Sjogren's syndrome (pSS patients), the majority of SEMA4D was detected in the sSEMA4D form (FIG. 1D). Quantitative analysis, as shown in FIGS. 1E and 1F, revealed that most of the SEMA4D in the normal salivary glands was in the mSEMA4D form, whereas in the salivary glands of patients with Sjogren's syndrome, most of the SEMA4D was present in the sSEMA4D form (FIGS. 1E and 1F).

Therefore, it was confirmed that in patients with Sjogren's syndrome, compared to the healthy control group, not only was the amount of SEMA4D in the salivary glands increased, but also the amount of sSEMA4D was significantly elevated.

### Example 2: Correlation Between CXCL12 Concentration and SEMA4D Concentration in Patients with Sjogren's Syndrome

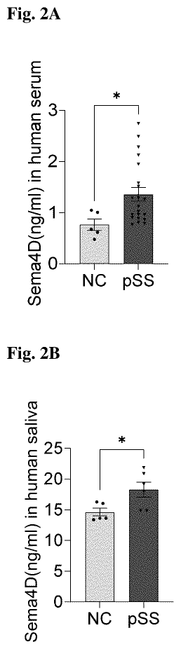

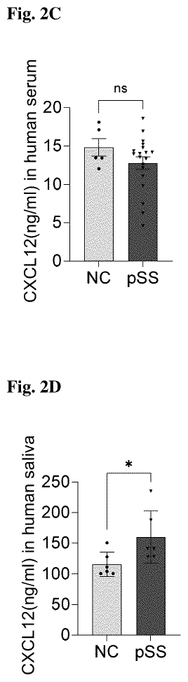

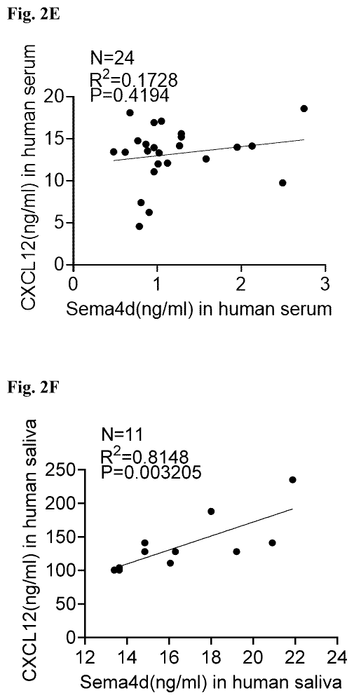

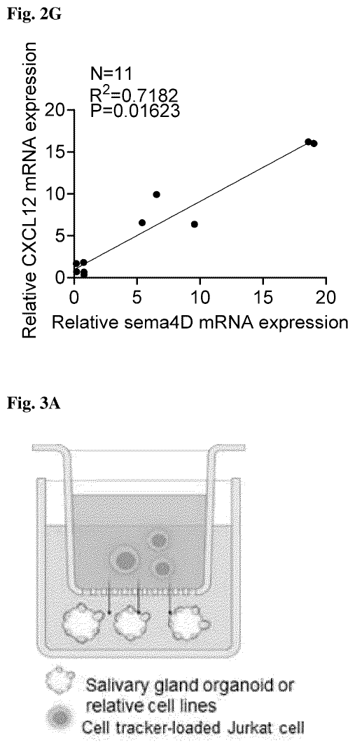

As described above, it was confirmed that the amount of sSEMA4D in the salivary glands of patients with Sjogren's syndrome was increased. Since sSEMA4D is known to be secreted extracellularly, the amount of sSEMA4D secreted into the saliva and serum of patients with Sjogren's syndrome (pSS) was analyzed and compared to the control group (NC). The results demonstrated that the levels of sSEMA4D in both the saliva and serum of patients with Sjogren's syndrome were significantly elevated compared to the control group (FIGS. 2A and 2B).

Next, the levels of CXCL12, a chemotactic factor for immune cells, were analyzed in the serum and saliva of patients with Sjogren's syndrome (pSS) in comparison to the control group (NC). The analysis revealed no significant difference in the serum levels of CXCL12 between the normal group and the patients with Sjogren's syndrome. However, it was found that the levels of CXCL12 in the saliva of patients with Sjogren's syndrome were significantly increased (FIGS. 2C and 2D).

A correlation analysis was performed between sSEMA4D and CXCL12 levels in the serum and saliva of patients with Sjogren's syndrome as described above. As a result, no significant correlation was observed between sSEMA4D and CXCL12 levels in the serum of the patients (FIG. 2E). However, a strong positive correlation was identified between sSEMA4D and CXCL12 levels in the saliva (FIG. 2F). Furthermore, the expression levels of SEMA4D and CXCL12 mRNA in the salivary glands of patients with Sjogren's syndrome were analyzed, and a statistically significant correlation between the two markers was also confirmed (FIG. 2G).

When both sSEMA4D and CXCL12 were used as biomarkers in saliva, the area under the receiver operating characteristic (ROC) curve (AUC) reached 0.967, whereas the AUCs for sSEMA4D alone and CXCL12 alone were 0.867 and 0.861, respectively. Accordingly, the simultaneous measurement of both sSEMA4D and CXCL12 in saliva provides improved diagnostic performance, exhibiting enhanced sensitivity and specificity for the diagnosis of Sjogren's syndrome.

| Sample | marker | AUC |
|---|---|---|
| saliva | sSEM4D | 0.867 |
| | CXCL12 | 0.861 |
| | sSEM4D + CXCL12 | 0.967 |
| serum | sSEM4D | 0.867 |
| | CXCL12 | 0.467 |
| | sSEM4D + CXCL12 | 0.867 |

Therefore, it was confirmed that the diagnostic accuracy for identifying patients with Sjogren's syndrome was superior when both sSEMA4D and CXCL12 levels in saliva were measured in combination, compared to measurement of sSEMA4D alone.

### Example 3: Relationship Between SEMA4D and Immune Cell Migration

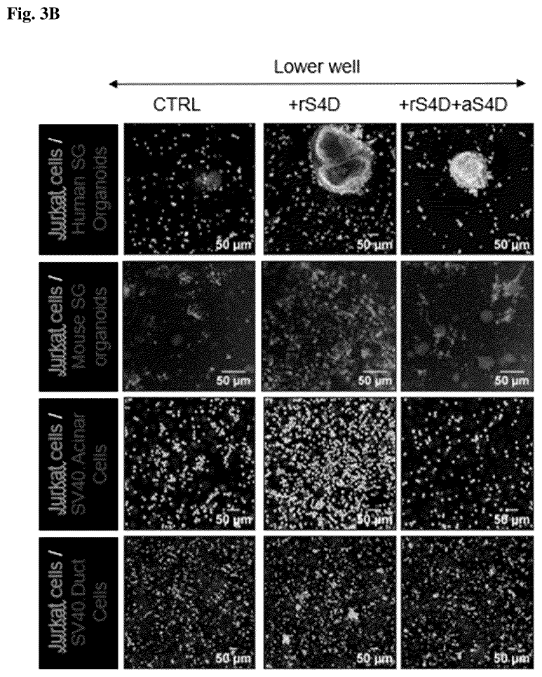

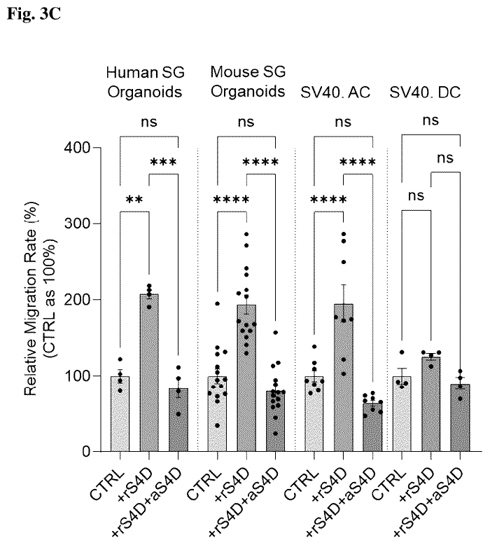

In order to reproduce the interaction between immune cells and salivary gland epithelial cells, a co-culture system was established as illustrated in FIG. 3A. This system utilized human salivary gland-derived ductal and acinar cell lines (SV40.Duct and SV40.Acinar cells, generously provided by Professor Yunjong Lee, Seoul National University Bundang Hospital), as well as mouse and human salivary gland-derived organoids, co-cultured with human CD4⁺ T cell lines (Jurkat cells), to simulate salivary gland epithelial tissues. Jurkat cells and salivary gland epithelial cells or tissues (organoids) were tagged with different fluorescent markers and seeded into the upper and lower chambers of a transwell system, respectively.

The lower chamber was treated with recombinant soluble SEMA4D (recombinant soluble/cleaved SEMA4D, +rS4D, R&D SYSTEMS, 7470-S4) or with both rS4D and an anti-SEMA4D blocking antibody (+aS4D). The recombinant SEMA4D used in this experiment was a cleaved, soluble form of the full-length SEMA4D protein produced by proteolytic cleavage.

For blocking SEMA4D activity, the anti-SEMA4D antibody BMA12 (eBioscience™) was employed in experiments using mouse cells, while the anti-human SEMA4D antibody sc-390675 (Santa Cruz Biotechnology) was used in experiments using human cells.

As shown in FIGS. 3B and 3C, treatment with recombinant SEMA4D (rS4D) led to an approximately twofold increase in the number of Jurkat cells that migrated to the lower chamber compared to the untreated control (CTRL). Co-treatment with rS4D and the anti-SEMA4D blocking antibody (aS4D) significantly suppressed immune cell migration, restoring it to levels similar to those observed in the control group. Notably, this migration-promoting effect was not observed in experiments involving the SV40.Duct cell line. These results indicate that rS4D promotes immune cell migration specifically in the presence of acinar cells, the salivary secretory cells of the salivary gland, and that this effect can be inhibited by the anti-SEMA4D blocking antibody (aS4D).

### Example 4: Relationship Between SEMA4D and Salivary Gland Epithelial Cells

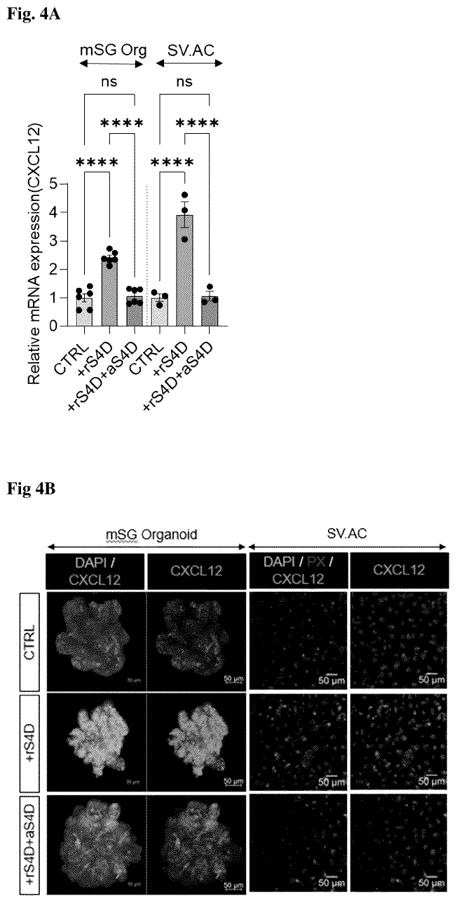

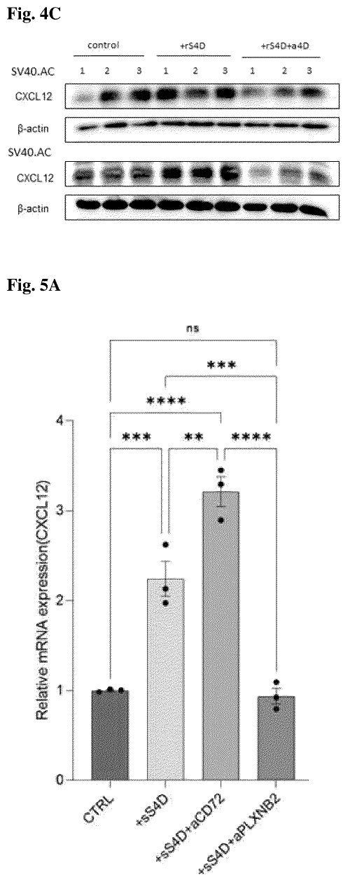

A human salivary gland epithelial cell line (SV40.AC) and a mouse salivary gland-derived organoid (mSG Organoid) were treated with recombinant SEMA4D (+rS4D) or both recombinant SEMA4D and an anti-SEMA4D blocking antibody (+rS4D+aS4D). Subsequently, the expression of CXCL12 was analyzed at the mRNA level (FIG. 4A), protein level (FIG. 4C), and cellular localization (FIG. 4B).

As illustrated in FIGS. 4A to 4C, it was confirmed that both the salivary gland epithelial cell line and the mouse salivary gland organoid showed an increase in CXCL12 production (mRNA, protein) by rS4D.

### Example 5: Identification of SEMA4D Receptors Related with CXCL12

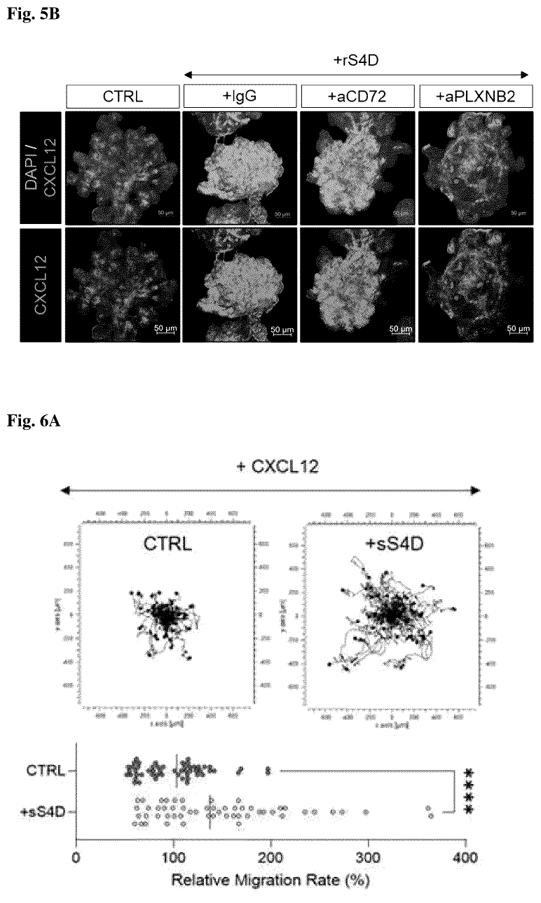

To identify the receptor responsible for mediating the observed response to recombinant SEMA4D (rS4D), receptor blocking experiments were conducted using antibodies targeting known SEMA4D receptors, including CD72 (monoclonal antibody, sc-25265, Santa Cruz Biotechnology) and Plexin B2 (monoclonal antibody, sc-373930, Santa Cruz Biotechnology). Mouse salivary gland-derived organoids were treated with rS4D in the presence or absence of each receptor-specific blocking antibody, and the expression levels of CXCL12 were subsequently analyzed. The results are presented in FIGS. 5A and 5B.

As shown in FIGS. 5A and 5B, treatment with rS4D resulted in increased CXCL12 expression at both the mRNA and protein levels. This upregulation was suppressed to baseline (control) levels by co-treatment with an anti-Plexin B2 blocking antibody (a-PLXNB2). In contrast, co-treatment with an anti-CD72 blocking antibody (aCD72) further enhanced CXCL12 expression. These findings indicate that sSEMA4D transmits its downstream signal primarily through Plexin B2 in salivary gland epithelial cells.

### Example 6: Confirmation of Effect of SEMA4D on T Cells

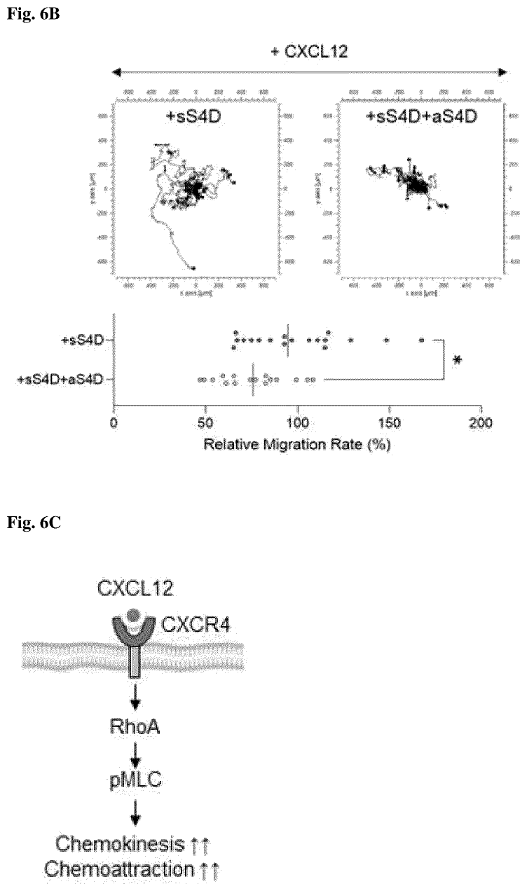

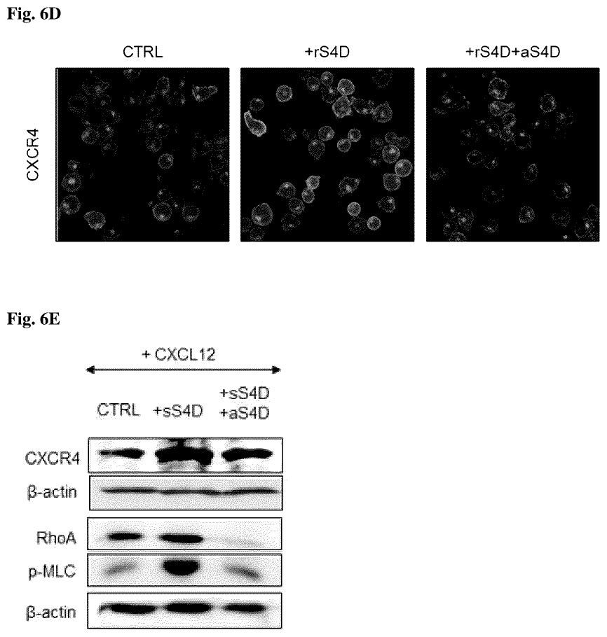

In order to investigate the effect of SEMA4D on T cell migration, Jurkat cells were treated with recombinant CXCL12, and the migration rate of the Jurkat cells was evaluated with or without the addition of recombinant SEMA4D (rS4D). The experimental results are illustrated in FIGS. 6A and 6B.

As shown in FIGS. 6A and 6B, treatment with recombinant SEMA4D (rS4D) resulted in a significant increase in the migration rate of Jurkat cells. This rS4D-induced enhancement in migration was suppressed to the control level upon co-treatment with an anti-SEMA4D blocking antibody (aS4D; sc-390675, Santa Cruz Biotechnology). There effects were presumed to be mediated through the CXCL12-CXCR4 signaling axis involving downstream activation of RhoA and phosphorylation of myosin light chain (pMLC), as depicted in FIG. 6C. Consistent with this, Jurkat cells treated with rS4D exhibited increased expression levels of CXCR4, RhoA, and pMLC. However, these elevated expression levels were reversed upon co-treatment with the anti-SEMA4D blocking antibody (FIGS. 6D and 6E). These results suggest that soluble SEMA4D secreted from the salivary glands of patients with Sjogren's syndrome not only promotes CXCL12 expression in the salivary gland epithelium to enhance T cell recruitment, but also upregulates CXCR4 expression in T cells, thereby increasing their responsiveness to CXCL12. Consequently, this dual mechanism may synergistically facilitate T cell infiltration and the formation of lymphocytic foci in the salivary glands.

### Example 7: Establishment of Sjogren's Syndrome Mouse Model

In order to determine the onset time point of Sjogren's syndrome-like symptoms, a non-obese diabetic (NOD) mouse model, widely recognized as a Sjogren's syndrome model, was employed.

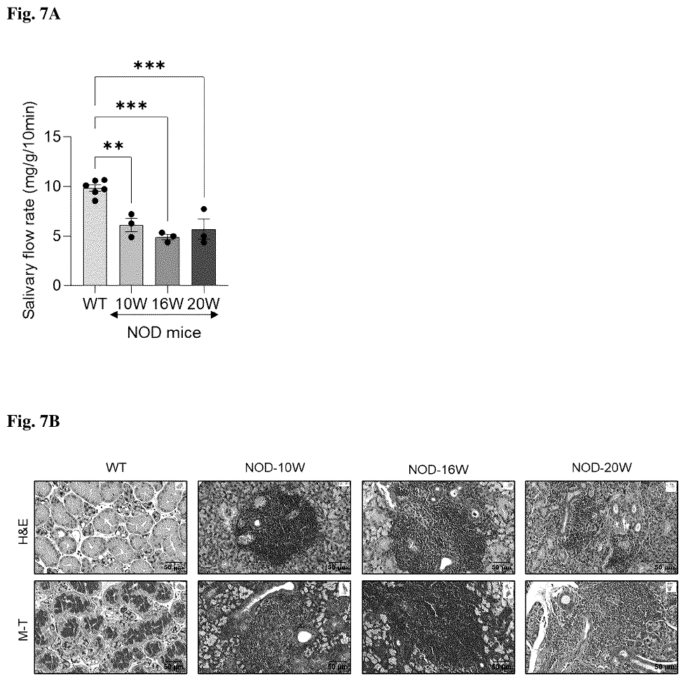

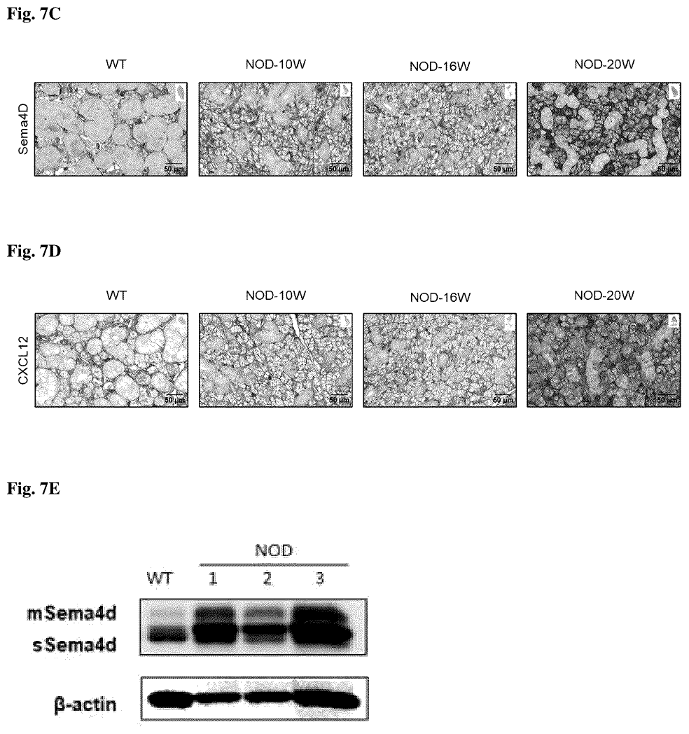

The NOD mice exhibited hallmark pathological features of Sjogren's syndrome, including lymphocytic infiltration into the salivary glands destruction, and reduced salivary secretion, beginning at approximately 9 to 10 weeks of age. The reduced saliva production persisted through 20 weeks of age (FIG. 7A). Correspondingly, lymphocytic foci began to appear in the salivary gland tissue at around 10 weeks of age (FIG. 7B). Furthermore, the protein expression levels of SEMA4D and CXCL12 in the salivary glands progressively increased (FIGS. 7C and 7D). Western blot analysis using an anti-SEMA4D antibody (sc-390675, Santa Cruz Biotechnology) and an anti-CXCL12 antibody (3740S, Cell Signaling Technology) demonstrated that both full-length membrane-bound SEMA4D (mSEMA4D) and the cleaved, secreted soluble SEMA4D (sSEMA4D) were elevated in 16-week-old NOD mice (FIG. 7E).

These findings confirm that, in the NOD mouse model, pathological changes similar to those observed in patients with Sjogren's syndrome—namely, increased expression of SEMA4D, sSEMA4D, and CXCL12, as well as immune cell infiltration into the salivary glands—were reproducibly observed from approximately 10 weeks of age.

### Example 8: Analysis of Administration Route for Treatment of Sjogren's Syndrome

Subsequently, the therapeutic potential of an anti-SEMA4D blocking antibody (anti-SEMA4D antibody; SC-390675, Santa Cruz) was evaluated in a Sjogren's syndrome animal model. To determine the optimal route of administration, the antibody was delivered either directly into the salivary glands or via intraperitoneal (IP) injection in 10-week-old NOD mice. Mice were sacrificed at 13 weeks of age, and salivary secretion and lymphocytic infiltration (Foci) in the salivary glands were assessed. For local administration, 1 to 1.2 mg of antibody was injected per salivary gland (administered into both salivary glands), based on mice weighing 20-25 g. For systemic administration, a single 108 mg dose was administered intraperitoneally.

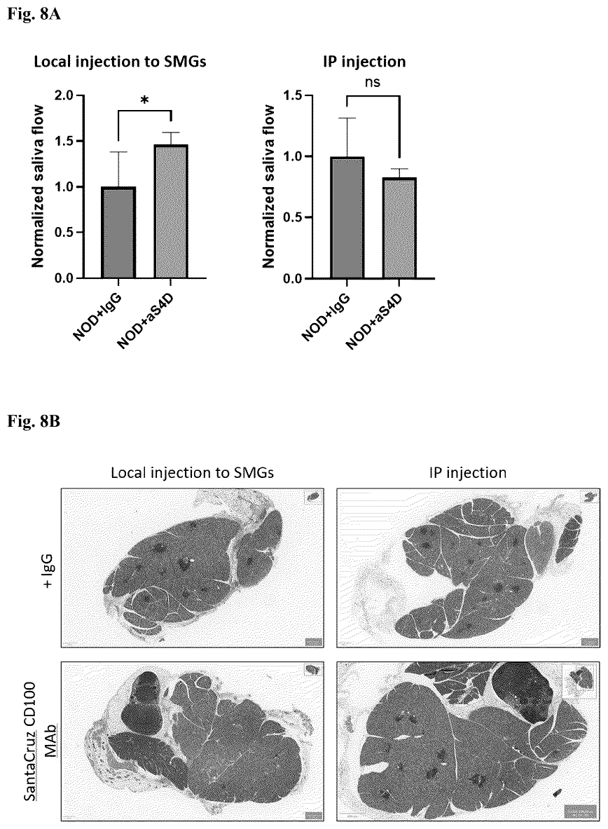

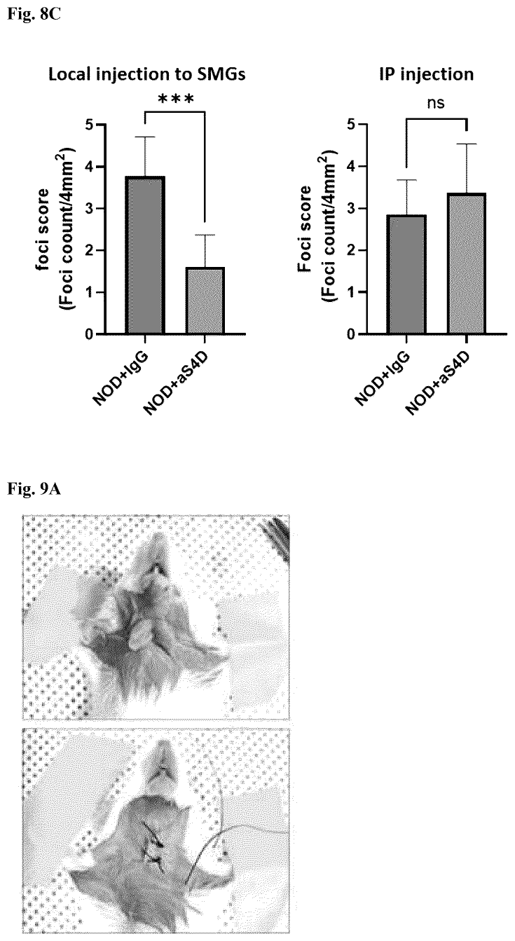

As shown in FIG. 8A, local administration of the anti-SEMA4D antibody into the salivary glands resulted in an average increase in salivary secretion of more than 50%, whereas intraperitoneal administration did not lead to a statistically significant change compared to the control group (treated with mouse IgG). Furthermore, histological analysis demonstrated that the number of Foci in the salivary glands was significantly reduced following local antibody administration, while no such reduction was observed in the intraperitoneally treated group (FIGS. 8B and 8C).

### Example 9: Results of Treatment of Sjogren's Syndrome by Anti-SEMA4D Blocking Antibody

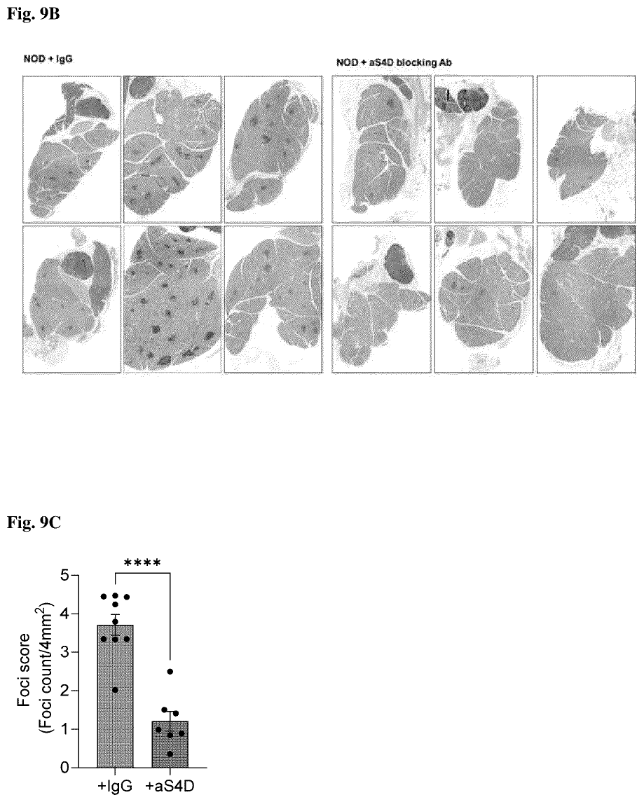

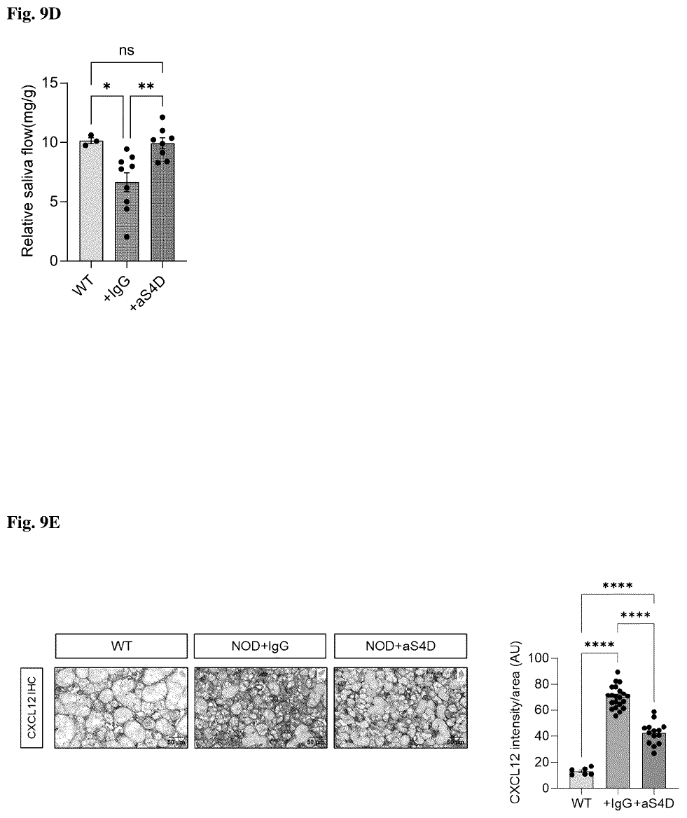

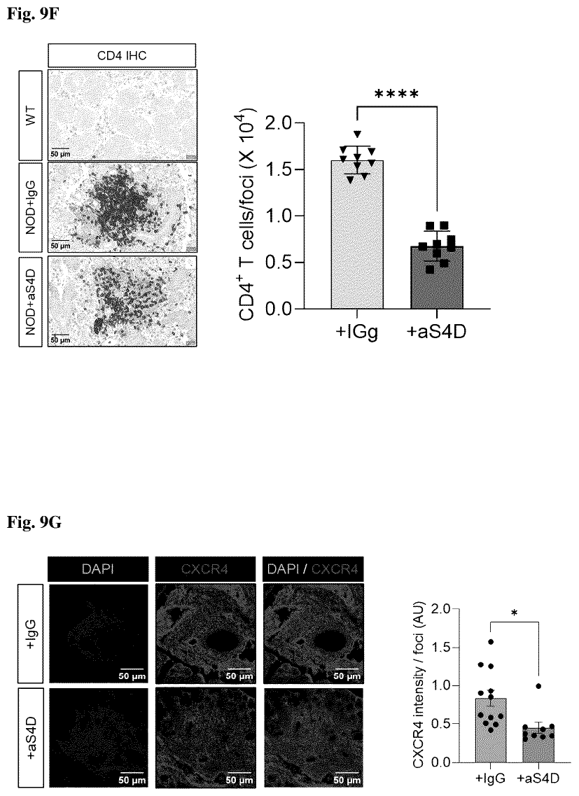

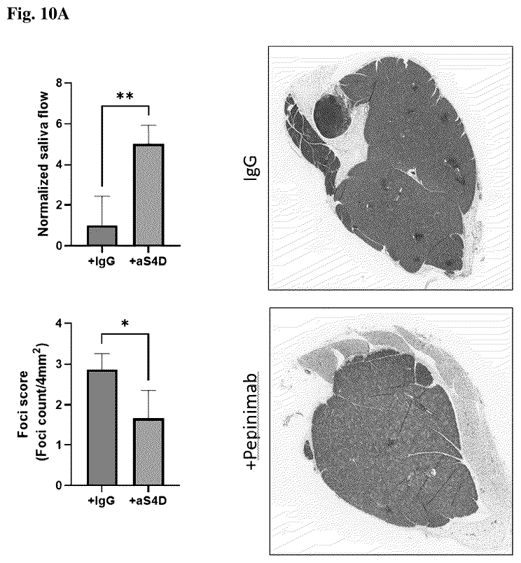

An anti-SEMA4D blocking antibody (anti-Sema 4D blocking antibody; SC-390675, Santa Cruz) or mouse IgG (control) was locally administered into the salivary glands of 10-week-old NOD mice. The animals were sacrificed at 13 weeks of age, and salivary gland tissue was harvested for analysis (FIG. 9A). The results are shown in FIGS. 9A to 9F.

As shown in FIGS. 9B and 9C, local administration of the anti-SEMA4D antibody (aS4D) effectively suppressed immune cell infiltration (Foci formation) in the salivary glands. Additionally, the reduced salivary secretion observed in the NOD mouse model was restored to a normal level following antibody treatment (FIG. 9D). The expression level of CXCL12 in the salivary glands was also significantly decreased in the antibody-treated group compared to the control group (FIG. 9E).

Furthermore, in the aS4D-treated group, the number of infiltrating CD4+ T cells in the salivary glands was reduced by more than 50% relative to the control, and the expression level of the CXCR4 protein, a receptor for CXCL12, was also markedly reduced (FIG. 9G).

### Example 10: Analysis of Treatment Results for Sjogren's Syndrome Using Various Antibodies

As described in Examples 8 and 9 above, the therapeutic efficacy of an anti-SEMA4D blocking antibody (anti-Sema 4D blocking antibody; SC-390675, Santa Cruz) was confirmed in a Sjogren's syndrome animal model. In addition, to further evaluate the therapeutic potential, other commercially available anti-SEMA4D antibodies were tested in the same model.

Specifically, Pepinemab, a humanized monoclonal antibody against SEMA4D, and BMA12 (BMA-12, eBioscience™), a rat-derived anti-SEMA4D antibody, were locally administered into the salivary glands of 10-week-old NOD mice. For each test group, corresponding control antibodies (human IgG and rat IgG, respectively) were used. At 13 weeks of age, saliva volume was measured and salivary glands were harvested to assess immune cell infiltration (Foci formation).

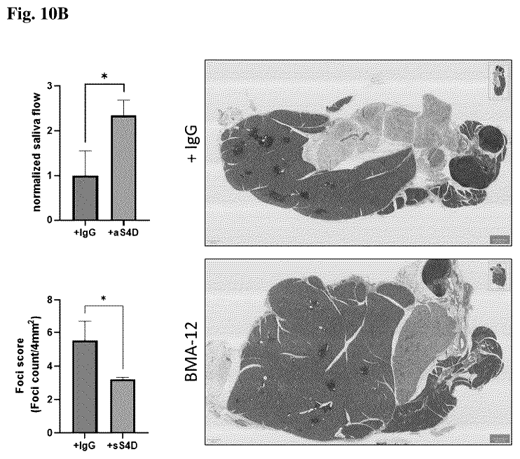

As shown in FIGS. 10A and 10B, local administration of both Pepinemab and BMA-12 significantly increased salivary secretion (by more than twofold) and markedly reduced the number of Foci in the salivary glands, confirming the therapeutic efficacy of the anti-SEMA4D antibodies.

While exemplary embodiments of the present disclosure have been described in detail above, it should be understood that the scope of the present disclosure is not limited thereto. Various modifications and alterations may be made by those skilled in the art based on the technical spirit of the present disclosure, and all such modifications fall within the scope of the present disclosure as defined by the appended claims.

Unless otherwise specifically defined, all technical terms used herein are intended to have the same meaning as commonly understood by those of ordinary skill in the art. The disclosures of all cited references and publications are incorporated herein by reference in their entirety.

1. A pharmaceutical composition for preventing or treating Sjogren's syndrome comprising an anti-SEMA4D blocking antibody as an active ingredient.

2. The pharmaceutical composition of claim 1, wherein the anti-SEMA4D blocking antibody is selected from the group consisting of a human antibody, a humanized antibody, a mouse antibody, a chimeric antibody, and a rat antibody.

3. The pharmaceutical composition of claim 2, wherein the anti-SEMA4D blocking antibody is a human antibody.

4. The pharmaceutical composition of claim 3, wherein the anti-SEMA4D blocking antibody is Pepinemab.

5. The pharmaceutical composition of claim 1, wherein the composition is formulated for administration by injection.

6. The pharmaceutical composition of claim 5, wherein the composition is administered locally into a salivary gland.

7. A diagnostic kit for Sjogren's syndrome, comprising an SEMA4D antibody as an active ingredient.

8. The diagnostic kit for Sjogren's syndrome of claim 7, further comprising:
a CXCL12 antibody.

9. The diagnostic kit of claim 7, wherein the diagnosis of Sjogren's syndrome is performed by measuring levels of soluble SEMA4D and CXCL12 in saliva obtained from a subject suspected of having Sjogren's syndrome.
